(19) United States
(12) Patent Application Publication
(10) Pub. No.: US 2018/0325336 A1
(43) Pub. Date: Nov. 15, 2018

(54) Toilet Seat Assembly with Ultraviolet Irradiating Elements

(71) Applicant: Thom Kai Chang, McKinney, TX (US)

(72) Inventor: Thom Kai Chang, McKinney, TX (US)

(21) Appl. No.: 15/934,922

(22) Filed: Mar. 23, 2018

Related U.S. Application Data
(60) Provisional application No. 62/504,465, filed on May 10, 2017.

(51) Int. Cl. A47K 13/30; A61L 2/10

(52) U.S. Cl. CPC A47K 13/302; A61L 2202/11; A61L 2/10

(57) ABSTRACT

A toilet seat assembly includes a contoured toilet seat for mounting over a toilet bowl, where the toilet seat has a reflective underside surface. The toilet seat assembly further includes a lid coupled to the toilet seat by a hinged coupling, where the lid also has a reflective underside surface. The toilet seat assembly includes an ultraviolet light ring encircling the toilet seat with a plurality of ultraviolet irradiating elements being operable to generate ultraviolet radiation that is directed downward into the toilet bowl and onto the toilet seat for disinfecting purposes.

### RELATED APPLICATION

This patent application claims the benefit of U.S. Provisional Patent Application No. 62/504,465 filed on May 2, 2017, incorporated herein by reference.

### FIELD

The present disclosure relates generally to sanitation devices, and in particular to a toilet seat assembly with ultraviolet (UV) irradiating elements.

### BACKGROUND

Toilets typically hosts a large number of germs, particularly toilets in public places. Studies have shown that pathogenic bacteria such as E. coli, Shigella, Staphylococcus, Salmonella, and Serratia marcescens, and viruses such as norovirus and influenza viruses can be found in typical toilets. What's more, experts say flushing the toilet creates an invisible cloud of “toilet plume” that's sent into the air by the gushing whirlpool in the toilet bowl. Besides potential transmission of diseases by aerosols, transmission via contact with contaminated toilet seat or toilet surfaces is also highly possible, especially in public and school settings. The dispersed aerosol from the toilet plume fall and adhere to surfaces such as toilet seat, door, door handle, toilet flush handle, faucet handle, sink, floor, etc. where users may come into contact. The toilet plume was first discovered in 1975 by microbiologist Charles Gerba who published a paper on the subject in the journal Applied and Environmental Microbiology. As recent as September 2013, an article on “Aerosol Generation by Modern Flush Toilet” published by Johnson et al. in the Journal of Aerosol Sci Tech examined possible bioaerosols generated by high efficient (HET), pressure-assisted high efficiency (PAT), and flushometer (FOM) toilets. It was shown that a flush produced by the highest-energy toilet could send up to 145,000 droplets with 95% <2 microns in diameter into the surrounding environment. Although few toilet plume-related illnesses have been documented, and the magnitude of this disease vector is unclear, some outbreaks of gastroenteritis on airplanes and cruise ships have been linked to this phenomenon, as well as a SARS outbreak in 2003 when a patient in Hong Kong may have spread the virus with a toilet flush. Besides transmission by aerosols, transmission via contacts with contaminated toilet seat or toilet surface is also highly possible, especially in school settings. In May of 2017, a norovirus outbreak grew from approximately 900 cases to more than 28,000 cases in the span of just a few days in Yolo County schools in northern California. The difficulties involved in ensuring good hand washing practices were cited as one of the probable causes for the outbreak. Accordingly, it is desirable to find solutions to disinfect the toilet seat and toilet bowl to minimize the microorganisms spread by the toilet plume and contact with contaminated surfaces.

### SUMMARY OF THE INVENTION

Described herein is a toilet seat assembly similar to a conventional toilet seat and cover, that is configured to automatically disinfect toilet seat and toilet bowl surfaces with ultraviolet (UV) light sources. The UV light elements are mounted on a ring encircling the toilet seat and irradiate the top surface of the toilet seat, the space between the underside of the toilet seat, bowl rim, and bowl. The seat cover is molded in a configuration that allows the housing of a battery on the topside and the maximum deflection of UV light on the chromium-coated underside. The power source can be a rechargeable battery in one version and from a power cord plugged into an electrical outlet in another version. The UV elements are turned on by a switch mechanism when the cover is in the down position and off when it is lifted up in the up position.

### BRIEF DESCRIPTION OF THE DRAWINGS

FIG. 1 is a perspective view of an exemplary embodiment of a toilet seat assembly with arrays of ultraviolet irradiating elements according to the teachings of the present disclosure;

FIG. 2 is another perspective view of an exemplary embodiment of a toilet seat assembly with arrays of ultraviolet irradiating elements according to the teachings of the present disclosure;

FIG. 3 is yet another perspective view of an exemplary embodiment of a toilet seat assembly with arrays of ultraviolet irradiating elements according to the teachings of the present disclosure;

FIG. 4 is a perspective view of an exemplary embodiment of a toilet seat according to the teachings of the present disclosure;

FIG. 5 is a perspective view of an exemplary embodiment of an ultraviolet irradiating element array for a toilet seat according to the teachings of the present disclosure;

FIG. 6 is another perspective view of an exemplary embodiment of a toilet seat assembly with arrays of ultraviolet irradiating elements showing a removable rechargeable battery pack according to the teachings of the present disclosure; and

FIGS. 7 and 8 are perspective views of an exemplary embodiment of the removable rechargeable battery pack for a toilet seat according to the teachings of the present disclosure.

### DETAILED DESCRIPTION

Due to the existence of abundant microorganisms in the toilet and the potential to spread these pathogens by the mere action of flushing and coming into contact with the toilet, it has become desirable to disinfect the toilet seat as well as toilet bowl surfaces to minimize or eliminate potential sources of illness and disease.

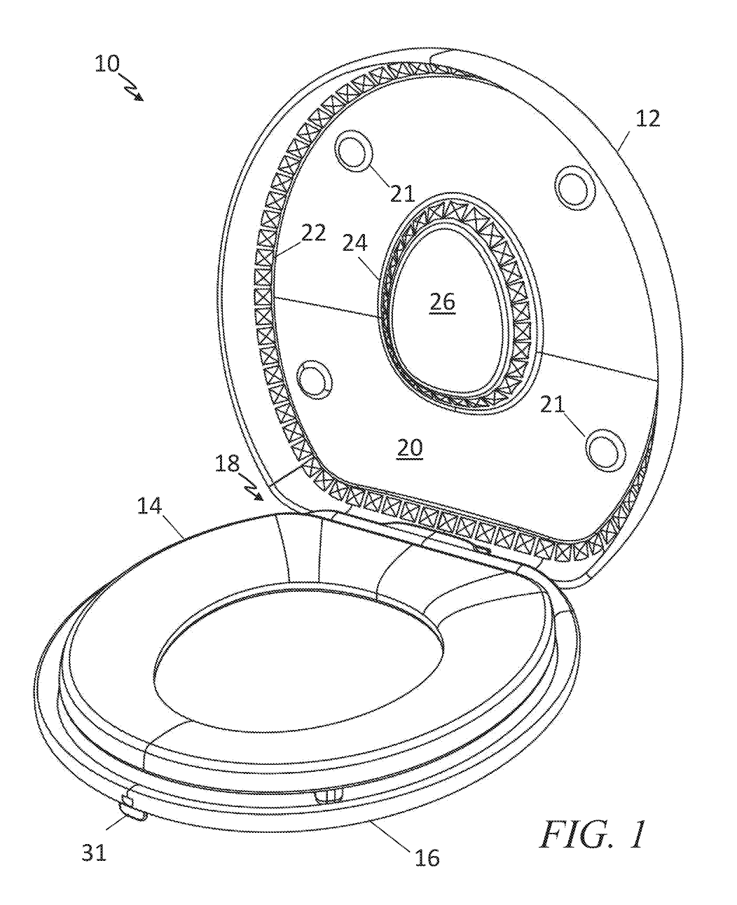

FIG. 1 is a perspective view of an exemplary embodiment of a toilet seat assembly 10 with arrays of ultraviolet irradiating elements according to the teachings of the present disclosure. The toilet seat assembly 10 is designed to be installed over a toilet bowl and includes three main components: a lid or cover 12, a seat 14, and an ultraviolet (UV) light ring 16. The lid 12, seat 14, and ultraviolet light ring 16 are connected at a hinge 18 with on/off switch mechanism mounted to the toilet (not shown) that enable these components to be placed in the up position or the down position. As shown in FIG. 1, the lid 12 is in the up position, and the seat 14 and UV light ring 16 are in the down position. The underside surface 20 of the toilet lid 12 is coated with a highly-reflective material, such as chrome-plating and the like to maximize reflectivity. Additionally, selected reflective regions 22 and 24 of the lid underside surface 20 are further faceted to maximize light dispersion. The facet pattern is designed to maximize light dispersion in one or more directions. As shown in FIG. 1, the ring with irradiating elements 16 frames the perimeter of the seat 14. The lid 12 further includes a contoured reflective region 26 encircled by faceted reflective region 24 that protrudes from the underside surface 20 and fits inside the seat opening. The reflective underside surface 20 of the lid 12 further includes a plurality of “feet” 21 that permit the lid 12 to rest upon the seat 14. The seat 14 further includes a plurality of “feet” 28 that serve as the interface between the seat 14 and the toilet bowl rim. A locking mechanism 31 allows the UV light ring to be latched onto the lid when it is in the down position and carried with the lid out of the way of the toilet seat when it is in the up position.

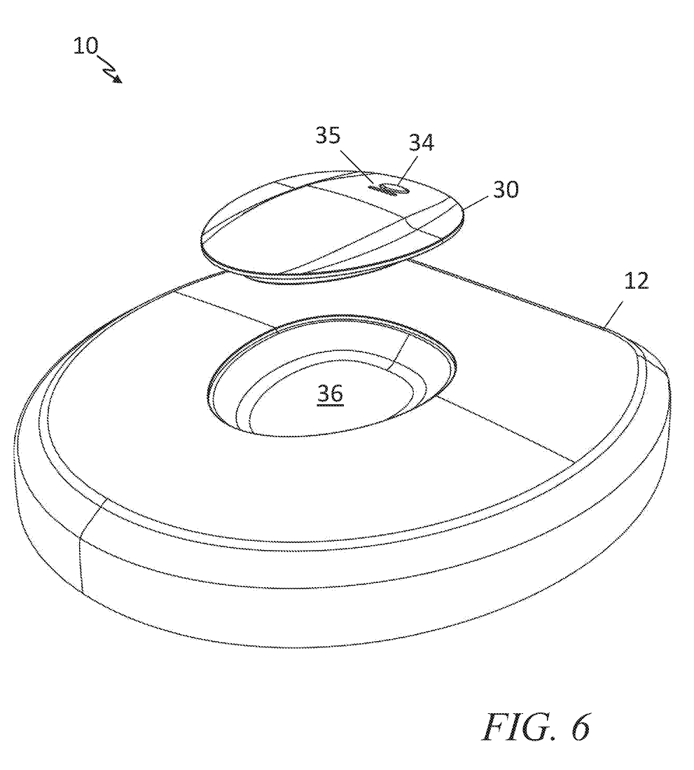

As shown in FIG. 1, the seat 14 and the UV light ring 16 are both in the down position disposed over the toilet bowl. The seat 14 is generally a contoured oval ring that sits over the toilet bowl and is designed to support the weight of a user. The seat 14 is disposed inside the UV light ring 16 that includes an array of UV light elements configured to direct UV radiation downward into the toilet bowl and onto top and bottom surfaces of the seat 14 with the aid of reflective surfaces 20 and faceted reflective regions 22 and 24. The UV light elements may comprise light bulbs or LEDs (light emitting diodes) and associated circuitry encased within a transparent casing with or without openings and may include reflective surfaces that help to focus and direct the generated UV light, for example. The UV light elements and associated circuitry may be powered by a rechargeable battery pack 30 (shown in FIGS. 6-8), or alternatively by AC power from a conventional power outlet. Preferably, the UV light elements are automatically switched on when the toilet lid is in the down position, and automatically shut off when the toilet lid is in the up position.

In an alternate embodiment, the UV light ring 16 further comprises non-UV light elements, such as LEDs of one or more colors that can be switched on and off according to a predetermined program or sequence, for example. These non-UV light elements are used for primarily aesthetic purposes.

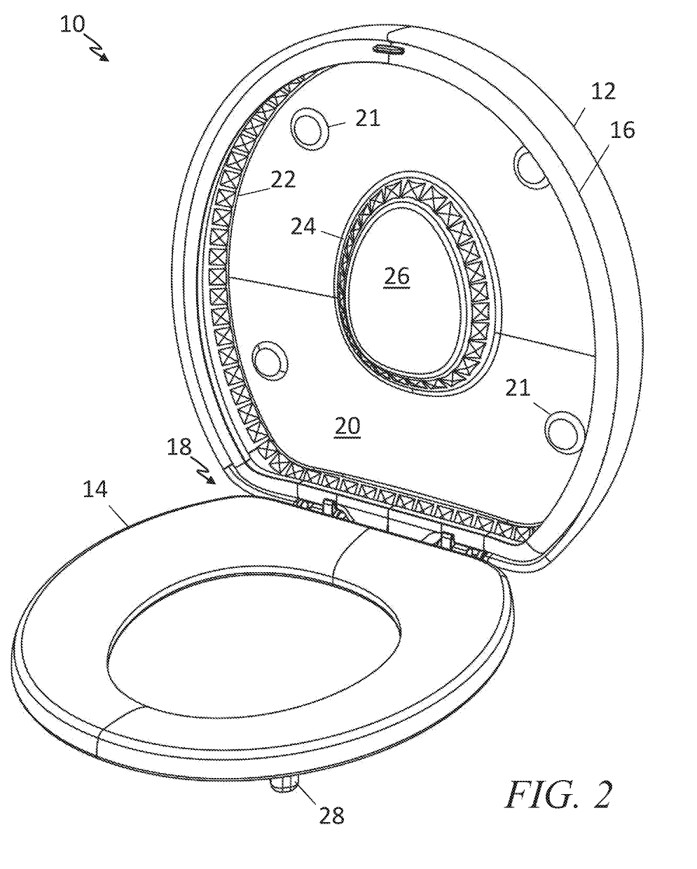

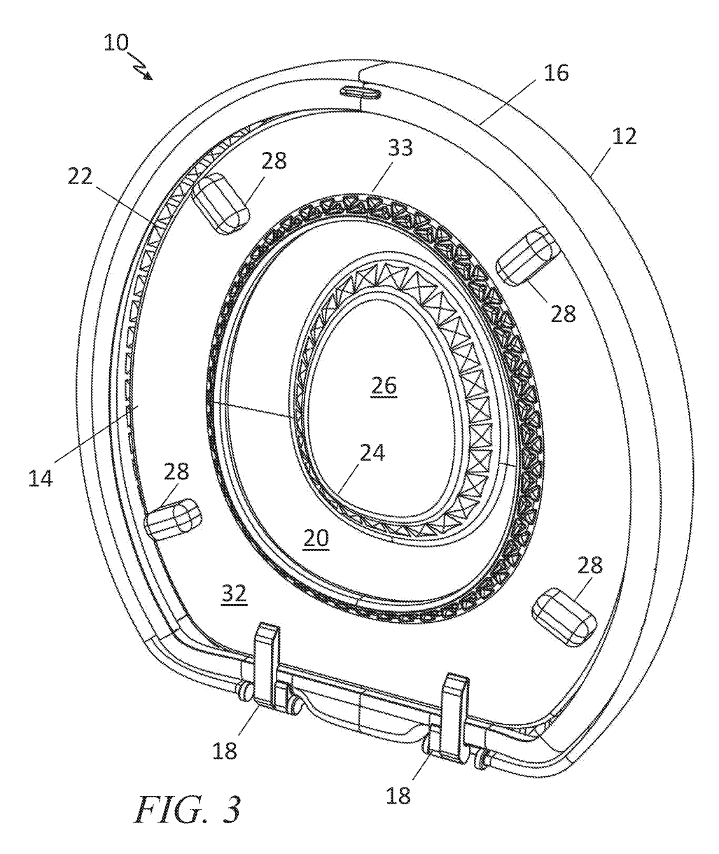

FIGS. 2 and 3 are additional perspective views of an exemplary embodiment of a toilet seat assembly with arrays of ultraviolet irradiating elements according to the teachings of the present disclosure. In FIG. 2, both the lid 12 and UV light ring 16 are in the up position, and the seat 14 is in the down position over the toilet bowl. In FIG. 3, the lid 12, seat 14, and UV light ring 16 are all in the up position. The underside surface 32 of the seat 14 is also preferably coated with a reflective material such as chrome to maximize light reflectivity into the toilet bowl. The underside surface 32 of the seat 14 also includes a perimeter ring of faceted reflective surfaces 33 that encircle the central opening of the seat 14. Preferably, the underside surface 32 of the seat 14 is contoured around its central opening to orient the faceted reflective region 33 for maximum light dispersion into the toilet bowl.

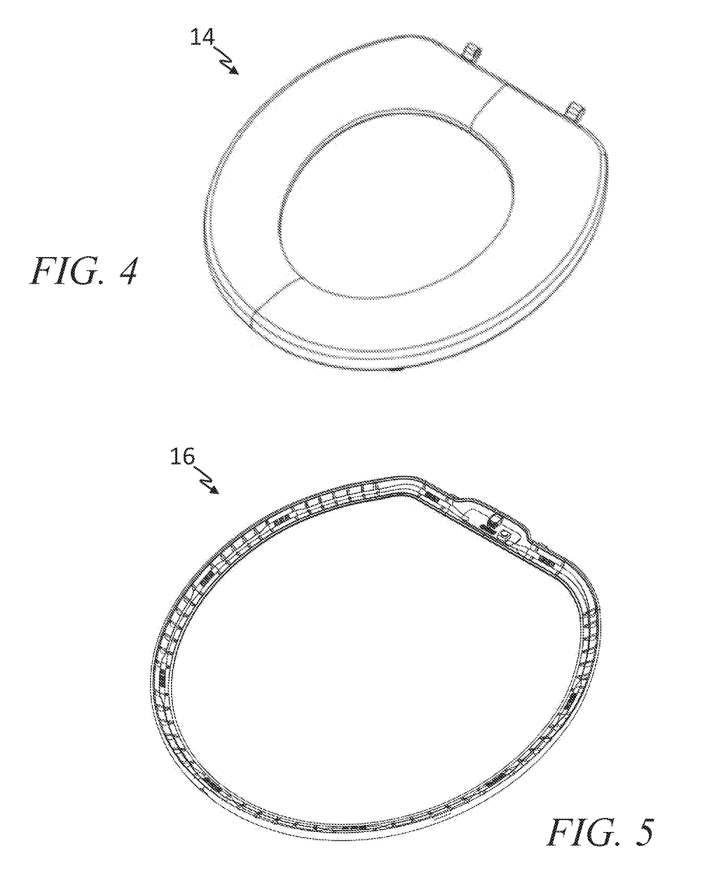

FIG. 4 is a perspective view of an exemplary embodiment of a toilet seat according to the teachings of the present disclosure, and FIG. 5 is a perspective view of an exemplary embodiment of an ultraviolet irradiating element array for a toilet seat according to the teachings of the present disclosure.

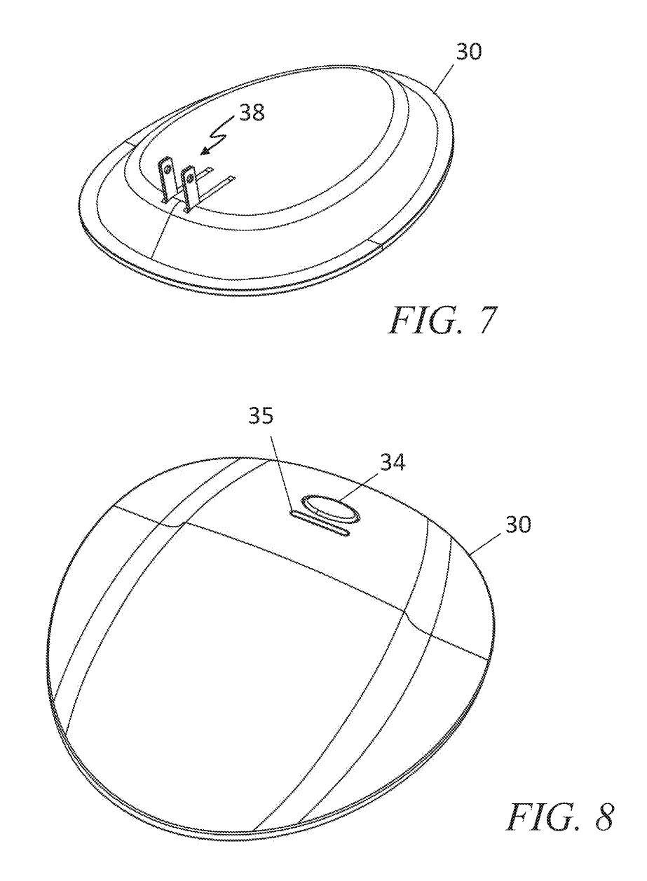

FIG. 6 is another perspective view of an exemplary embodiment of a toilet seat assembly with arrays of ultraviolet irradiating elements showing a removable rechargeable battery pack 30 according to the teachings of the present disclosure. The battery pack 30 includes a status light (LED) 35 that may indicate the charging and/or operating status of the light ring 16 and battery pack 30. The battery pack 30 is preferably contoured to be disposed and housed within a recess 36 defined in the top surface of lid 12. A release switch 34 is also provided which enables a user to release the battery pack from the recess 36 and snap it back into place. As shown in FIGS. 7 and 8, the battery pack 30 includes an AC power plug 38 that enable the battery pack to be removed from the lid 12 to be recharged using a conventional power outlet. Alternatively, the battery pack 30 can be recharged in place by using a power cord.

In operation, when the lid 12, seat 14, and UV light ring 16 are in the down position over the toilet bowl, the reflective surfaces and faceted reflective regions help to direct UV radiation into the toilet bowl and onto toilet seat surfaces. The short-wavelength UV radiation kills microorganisms and disinfects the surfaces of the toilet bowl and toilet seat to greatly diminish potential sources of illness and disease.

It should be noted that in alternate embodiments, the UV irradiating elements may be incorporated in the underside surface of the lid and/or the seat to maximize the UV radiation reaching all surface areas of the toilet bowl and seat.

The features of the present invention which are believed to be novel are set forth below with particularity in the appended claims. However, modifications, variations, and changes to the exemplary embodiments of a toilet seat assembly with ultraviolet irradiating elements described above will be apparent to those skilled in the art, and the described herein thus encompasses such modifications, variations, and changes and are not limited to the specific embodiments described herein.

What is claimed is:

1. A toilet seat assembly comprising:
a contoured toilet seat for mounting over a toilet bowl, the toilet seat having a reflective underside surface;
a lid coupled to the toilet seat by a hinged coupling, the lid having a reflective underside surface; and
an ultraviolet light ring encircling the toilet seat and having a plurality of ultraviolet irradiating elements, the ultraviolet light ring being coupled to the toilet seat by the hinged coupling, the plurality of ultraviolet irradiating elements being operable to generate ultraviolet radiation.

2. The toilet seat assembly of claim 1, wherein the lid further comprises faceted reflective regions in its reflective underside surface.

3. The toilet seat assembly of claim 1, wherein the lid further comprises a faceted outer perimeter reflective region and a faceted inner perimeter reflective region in its reflective underside surface.

4. The toilet seat assembly of claim 1, further comprising a rechargeable battery.

5. The toilet seat assembly of claim 1, further comprising a removable rechargeable battery pack disposed within a contoured recess defined in a top surface of the lid.

6. The toilet seat assembly of claim 1, wherein the rechargeable battery pack further comprises a status indicator.

7. The toilet seat assembly of claim 1, wherein the plurality of ultraviolet irradiating elements are operable to automatically generate and direct ultraviolet radiation into the toilet bowl and onto surfaces of the toilet seat when the toilet lid is in the down position, and further operable to be automatically shut off when the toilet lid is in the up position.

8. A toilet seat assembly comprising:
a toilet seat for mounting over a toilet bowl, the toilet seat having a reflective underside surface;
a lid coupled to the toilet seat by a hinged coupling, the lid having a reflective underside surface where selected regions thereof are faceted; and
a plurality of ultraviolet irradiating elements coupled to the lid and toilet seat, the plurality of ultraviolet irradiating elements being operable to automatically generate and direct ultraviolet radiation onto surfaces of the toilet seat and toilet bowl when the toilet lid is in the down position, and further operable to be automatically shut off when the toilet lid is in the up position.

9. The toilet seat assembly of claim 8, wherein the lid further comprises a faceted outer perimeter reflective region and a faceted inner perimeter reflective region in its reflective underside surface.

10. The toilet seat assembly of claim 9, wherein the lid further comprises a contoured reflective region encircled by the faceted inner perimeter and protruding into a central opening of the seat.

11. The toilet seat assembly of claim 8, further comprising a removable rechargeable battery pack disposed within a contoured recess defined in a top surface of the lid.

12. The toilet seat assembly of claim 11, further comprising a releasable latch securing the rechargeable battery pack within the contoured recess.

13. The toilet seat assembly of claim 8, wherein the rechargeable battery pack further comprises a status indicator.

14. A toilet seat assembly comprising:
a toilet seat for mounting over a toilet bowl, the toilet seat having a reflective underside surface;
a lid coupled to the toilet seat by a hinged coupling, the lid having a reflective underside surface;
a plurality of ultraviolet irradiating elements coupled to at least one of the lid and toilet seat, the plurality of ultraviolet irradiating elements being operable to generate ultraviolet radiation directed into the toilet bowl and onto surfaces of the toilet seat for disinfecting purposes; and
a rechargeable battery pack disposed within a contoured recess defined in a top surface of the lid.

15. The toilet seat assembly of claim 14, further comprising a releasable latch securing the rechargeable battery pack within the contoured recess.

16. The toilet seat assembly of claim 14, wherein the rechargeable battery pack further comprises a status indicator.

17. The toilet seat assembly of claim 14, wherein the lid further comprises faceted reflective regions in its reflective underside surface to maximize UV radiation dispersion.

18. The toilet seat assembly of claim 14, further comprising a locking mechanism configured to latch the plurality of ultraviolet irradiating elements onto the lid.

19. The toilet seat assembly of claim 14, wherein the plurality of ultraviolet irradiating elements is operable to automatically generate and direct ultraviolet radiation into the toilet bowl and onto surfaces of the toilet seat when the toilet lid is in the down position, and further operable to be automatically shut off when the toilet lid is in the up position.
